(19) United States
(12) Patent Application Publication
(10) Pub. No.: US 2016/0100982 A1
(43) Pub. Date: Apr. 14, 2016

(54) ULTRASONIC VITRECTOMY NEEDLE

(71) Applicant: Bausch & Lomb Incorporated, Rochester, NY (US)

(72) Inventor: Brian D. McCary, Clayton, MO (US)

(21) Appl. No.: 14/513,459

(22) Filed: Oct. 14, 2014

(51) Int. Cl. A61F 9/007

(52) U.S. Cl. CPC A61F 9/00745; A61M 1/008

(57) ABSTRACT

A needle for use with an ultrasonic handpiece for dissecting and aspirating vitreous tissue includes a cannula having a lumen extending from a port adjacent a cannula distal tip to a cannula proximal end. The cannula port has a cross-sectional area less than a cross-sectional area of the cannula lumen. A hub attached to the cannula proximal end attaches to the ultrasonic handpiece. A needle lumen is formed from the cannula port to a proximal end of the hub. A length of the needle lumen is approximately an odd quarter of a drive frequency wavelength of the ultrasonic handpiece and a cannula length measured from the cannula distal tip to a distal end of the hub is long enough to extend from an incision site across a posterior segment of an eye.

### FIELD

The present disclosure relates to ophthalmic ultrasonic handpieces that include aspiration of dissected tissue. More particularly, the present disclosure relates to aspirating ultrasonic handpieces for removal of vitreous from the posterior chamber of an eye.

### BACKGROUND

This section provides background information related to the present disclosure which is not necessarily prior art.

An ultrasonic device for the removal of vitreous has been previously described in U.S. patent application Ser. No. 14/020,386 filed on 6 Sep. 2013, entitled Vibrating Surgical Device for Removal of Vitreous and Other Tissue, published as US2014/0074013; the entire disclosure of the forgoing application is incorporated by reference. The previous patent application discloses a device having an ultrasonically driven needle with a distal end having a port that is smaller than a lumen of the needle. Vitreous is disrupted or liquefied across the port when a bi-directional flow of tissue through the port is created without creating cavitation externally of the distal end.

Additional construction details of the needle to be vibrated have been identified, ensuring efficient operation of the device and avoiding certain low-efficiency construction and operation parameters.

### DRAWINGS

The drawings described herein are for illustrative purposes only of selected embodiments and not all possible implementations, and are not intended to limit the scope of the present disclosure.

FIG. 1 is an elevation of a needle according to an exemplary embodiment;

FIG. 2 is an cut away view of FIG. 1 taken along line 2-2;

FIG. 3 is a cut away view of another exemplary embodiment;

FIG. 4 is a partial cut away view of a yet another exemplary embodiment; and

FIG. 5 is a graphical representation of simulated displacement flow in a needle.

Corresponding reference numerals indicate corresponding parts throughout the several views of the drawings.

### DETAILED DESCRIPTION

Example embodiments will now be described more fully with reference to the accompanying drawings.

In subsequent development efforts it has been found that some needle lengths appeared to work better than others. Mathematical simulations were performed and showed that, at certain drive frequencies, the water column of the needle lumen may resonate in a manner reducing an apparent inertial impedance of the water column at a distal needle tip and this apparent impedance is parasitic to the bi-directional reciprocating flow through the needle port. This reduction of apparent inertial impedance occurs when the overall distance of the water column is an even number of quarter wavelengths, λ, (in water) of the drive frequency, e.g. ½ λ, λ, 1.5 λ, etc. Conversely, it was found that selecting the drive frequency so that the overall length of the water column is approximately an odd number of quarter wavelengths, e.g. ¼ λ, ¾ λ, 1.25 λ, etc., avoids this reduction of apparent impedance condition and ensures the desired bi-directional flow across the needle port without external cavitation of the distal end.

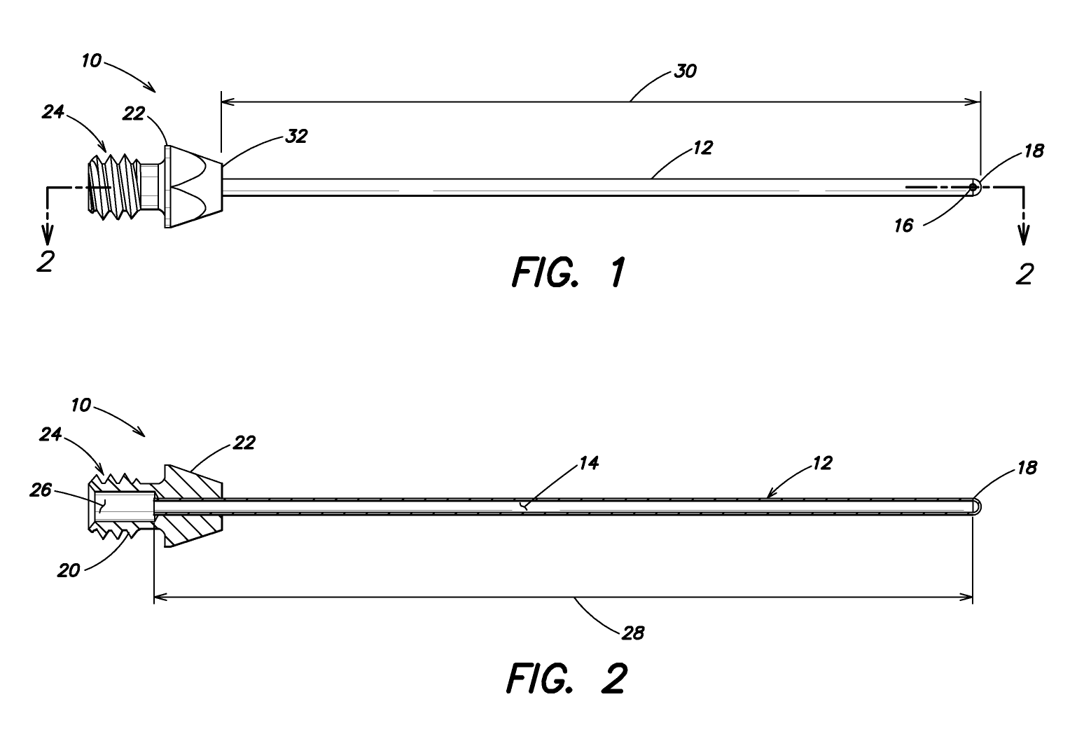

FIGS. 1 and 2 show the difference between a cannula length and a needle lumen length. A needle 10 is for use with an ultrasonic handpiece (not shown) for dissecting and aspirating vitreous tissue. The needle 10 includes a cannula 12 having a lumen 14 extending from a port 16 adjacent a cannula distal tip 18 to a cannula proximal end 20. The cannula port 16 has a cross-sectional area less than a cross-sectional area of the cannula lumen 14. A hub 22 may be attached to the cannula proximal end 20, as shown. The hub 22 may include structure 24 for attachment to the unshown ultrasonic handpiece. In the embodiments shown the hub attachment structure 24 are threads but could be any acceptable attachment structure that would adequately transmit ultrasonic vibration from the handpiece through the needle 10. The hub 22 includes a lumen, shown generally at 26, extending through the hub 22. The hub lumen 26 is coaxial and in communication with the cannula lumen 14. The hub 22 may be attached to cannula 12 by any acceptable means such as press fitting, welding, adhesives, or the like.

A length 28 of the cannula lumen is taken from the port 16 to the cannula proximal end 20. Further, the cannula lumen length is approximately an odd quarter of a drive frequency wavelength of the ultrasonic handpiece and a cannula length 30 measured from the cannula distal tip 18 to a distal end 32 of the hub 22 is long enough to extend from an incision site across a posterior segment of an eye (not shown).

Examples of cannula lumen lengths and drive frequencies will be described further below. Cannula length 30 should be long enough to extend through an entry site alignment device (not shown) and across the posterior segment of an eye without the handpiece causing any undo spike in intraocular pressure from pressing against the exterior of the eye. In most instances a cannula length 30 of approximately 31-33 millimeters (mm) will be sufficient. However, some eyes and some surgeons may prefer longer cannula lengths of 38 mm or more. It is noted that because of the outer diameter of the cannula 12 is necessarily small, e.g. 23, 25, or 27 gauge (ga.), there will be a trade-off between a cannula's stiffness and its length. The longer a cannula's length the less stiff it will be with a greater potentially for breaking compared to a shorter cannula length of the same outer diameter and wall thickness (the distance between an outer cannula 12 surface and an inner surface defining lumen 14).

The desired water column length to avoid the apparent impedance problem identified above, for FIG. 2 is defined by the cannula lumen length 28 because the transition from cannula proximal end 20 is to the hub lumen 26 that is significantly larger than the cannula lumen 14. A hub lumen twice the diameter of the cannula lumen is certainly significantly larger than the cannula lumen, as the average velocity in the hub lumen will be 25% of the average velocity in the cannula lumen, and the resulting kinetic energy of the moving material in the hub lumen will only be 6.25% of the energy in the cannula lumen—a significant difference. A 10% change in diameter is probably not significant, as the velocity in the hub lumen will still be 80% of the velocity in the cannula lumen, and the resulting kinetic energy in the hub region will still be 64% of the kinetic energy in the cannula. At a point in between these two extremes, for instance at a hub lumen diameter 1.5 times the cannula lumen diameter, velocity will have dropped by at least 50%, and energy by 80%. The water column length may therefore be considered to be the distance along the axis of the cannula from the port to a point that the diameter of the path is about 50% larger than the original cannula diameter at the port location. This larger diameter must be of sufficient length to reduce the energy in the water column, for example a distance of multiple hub lumen diameters past the initial transition point from a small lumen diameter to a large lumen diameter. Radiuses or tapers at the hub end of the lumen can therefore be accounted for with this definition; the termination of the water column will be at a distance about 1.5× the largest lumen diameter from the point on the taper or hub where the transition from the smallest lumen diameter begins. A short transition from a small diameter to a larger diameter and back to a smaller diameter somewhere along the cannula will not define the end of the water column. In needle constructions where the needle is not straight, the water column length will be the distance along the internal flow path axis from the port to the large lumen diameter, typically located at the needle hub.

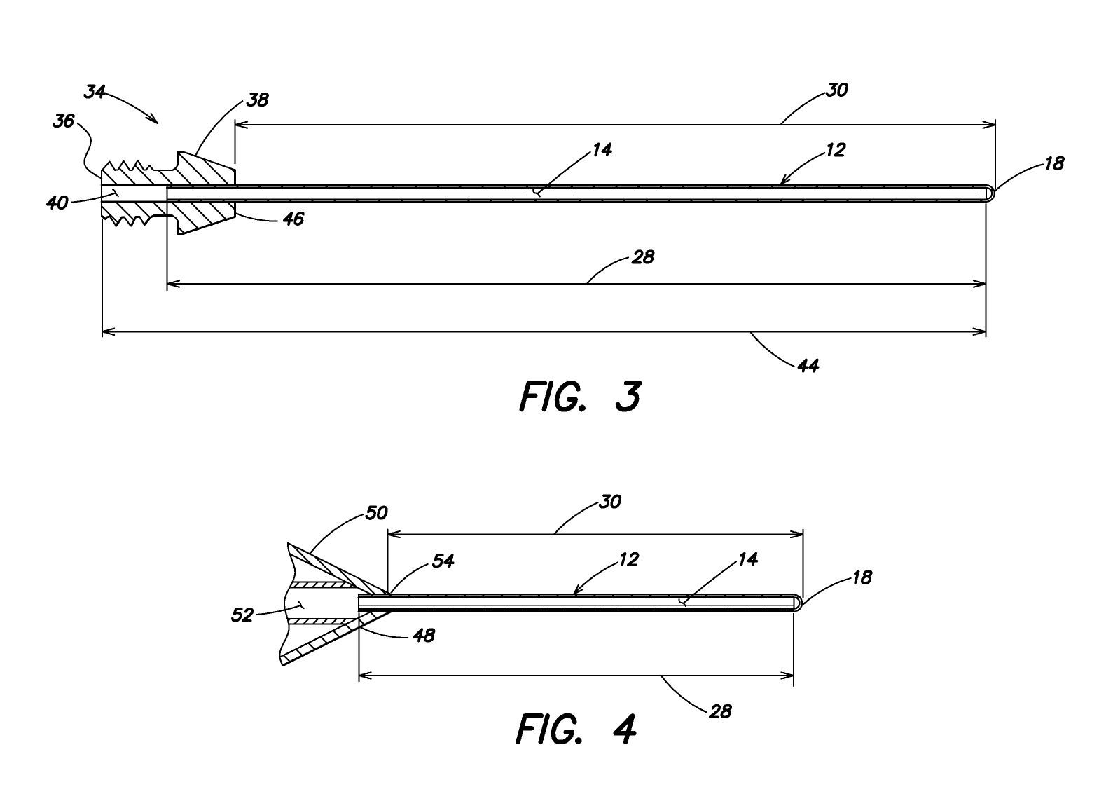

For example, the embodiment of FIG. 3 requires the desired water column length of needle 34 to be measured such that a needle lumen is formed from the cannula port 16 to a proximal end 36 of a hub 38. The needle lumen is comprised of cannula lumen 14 and a lumen 40 of hub 38. A length 44 of the needle lumen in this embodiment is the desired water column length because the lumen 40 is only slightly larger than cannula lumen 14. In this embodiment, lumen 40 is only larger than cannula lumen 14 by an amount to achieve a press or frictional connection with cannula 12. Similar to the cannula lumen length 28 described above, a length 44 of the needle lumen is approximately an odd quarter of a drive frequency wavelength of the unshown ultrasonic handpiece. The cannula length 30 of this embodiment is the same as for FIG. 2 and measured from the cannula distal tip 18 to a distal end 46 of the hub 38. The cannula length 30, as with the embodiment of FIG. 2, is long enough to extend from an incision site across a posterior segment of an eye (not shown).

In a further example embodiment, the cannula 12, of FIG. 4, is for attachment to a distal end 48 of an ultrasonic handpiece 50 (shown in a partial cut-away elevation), such that the cannula lumen 14 is in communication with an aspiration path 52 formed in the ultrasonic handpiece 50. The aspiration path 52 has a cross-sectional area significantly larger than the cannula lumen cross-sectional area. Therefore, the desired water column length can be taken as the same as the cannula lumen length 28. The length 28 of the cannula lumen 14 again is approximately an odd quarter of a drive frequency wavelength of the ultrasonic handpiece 50 and the cannula length 30 is measured from the cannula distal tip 18 to the distal end 54 of the ultrasonic handpiece 50. The cannula length 30 is long enough to extend from an incision site across a posterior segment of an eye (not shown).

As will be described below, the drive frequency of the ultrasonic vibration from the handpiece will determine the desired water column length. The water column length, as described above, may be the cannula lumen length 28 or it may be a different length depending on the form factor of the needle used. Conversely, if a desired cannula length is known and the form factor details of a needle's construction is known, then desirable drive frequencies for such a needle can be determined.

Selecting the drive frequency for a given needle is based on the length of the water column measured from the needle port to a location where there is a transition from the cannula lumen to a significantly larger diameter aspiration path portion. This transition from a small cannula lumen to a significantly larger diameter aspiration path portion minimizes any residual acoustic effects of the significantly larger diameter aspiration path portion and essentially simplifies the desired cannula or needle lumen calculations by assuming that the transition to the significantly larger diameter aspiration path portion is a transition into infinite space. As will be shown below, an acceptable water column length may be achieved by avoiding the use of a frequency for which the relevant lumen length is any multiple of a half wavelength of the frequency in water. Further, the optimal water column length is achieved by selecting a frequency such that the water column length is an odd multiple of an odd quarter wavelength of the frequency, in water.

The frequency and wavelength of a wave in a medium are related to the speed of the wave in the medium by the well-known equation:

λ*f=c (1)

Where λ is the wavelength of the wave in the medium, f is the frequency of the wave in the medium, and c is the phase speed of the wave in the medium.

For water at standard temperature and pressure, the phase speed of acoustic waves is known to be approximately 1500 meters per second (m/s), equivalent to 1,500,000 millimeters per second (mm/sec).

For a given water column length, frequencies to avoid will therefore be those for which,

{m/2}*λ=l, (2)

where l is the water column length (typically slightly longer that the external length of the lumen), and m is any positive integer (1(half wavelength), 2(full wavelength), 3(1.5 wavelengths . . . ). In most embodiments, the water column length will be equal to one of the cannula lumen length 28 or a needle lumen length 44 as described above. However, shorter or longer water column lengths can be achieved depending on the location of the transition from the cannula lumen to a significantly larger aspiration path portion.

Combining equations (1) and (2) above to eliminate λ, gives the equation:

f_{avoid}=(c/l)*{m/2} (3)

Using equation 3 for a cannula lumen length 1 of 38 mm (which permits 5 to 10 mm of tube length for joining the cannula to the handpiece or a hub) and the speed of 1,500,000 mm/sec identified above, (c/l)=39474 hertz (Hz), or about 40 kHz, and two f_{avoid} frequencies will be about 20 kHz (m=1) and 40 kHz (m=2).

Using the same methodology, optimal drive frequencies to use will be those for which,

{(2n−1)/4}*λ=l, (4)

where l is the water column length or for practical purposes the cannula lumen length or needle lumen length as discussed above, and n is any positive integer (1, 2, 3 . . . ). In practice, n will typically be a small integer, such as 1 or 2, so that the cannula length is sufficient and the cannula is stiff and durable enough to withstand use in an operation without breaking.

Combining equations (1) and (4) above to eliminate λ, gives the equation:

fₒₚₜᵢₘₐₗ=(c/l)*{(2n−1)/4} (5)

Using equation 5 for a cannula or needle lumen length of 1 of 38 mm and c is approximately the phase speed of an acoustic wave in water (1,500,000 mm/sec identified above), (c/l)=39474 Hz, or about 40 kHz, and two fₒₚₜᵢₘₐₗ drive frequencies will be about 10 kHz (n=1, a quarter wavelength) and 30 kHz (n=2, three-quarters of the wavelength).

Equation 5 can be rearranged to express a desired cannula or needle lumen length as:

l=(c/f)*{(2n−1)/4} (6)

where, again, c is approximately a phase speed of an acoustic wave in water, l is the needle lumen length, f is the drive frequency, and n is a positive integer.

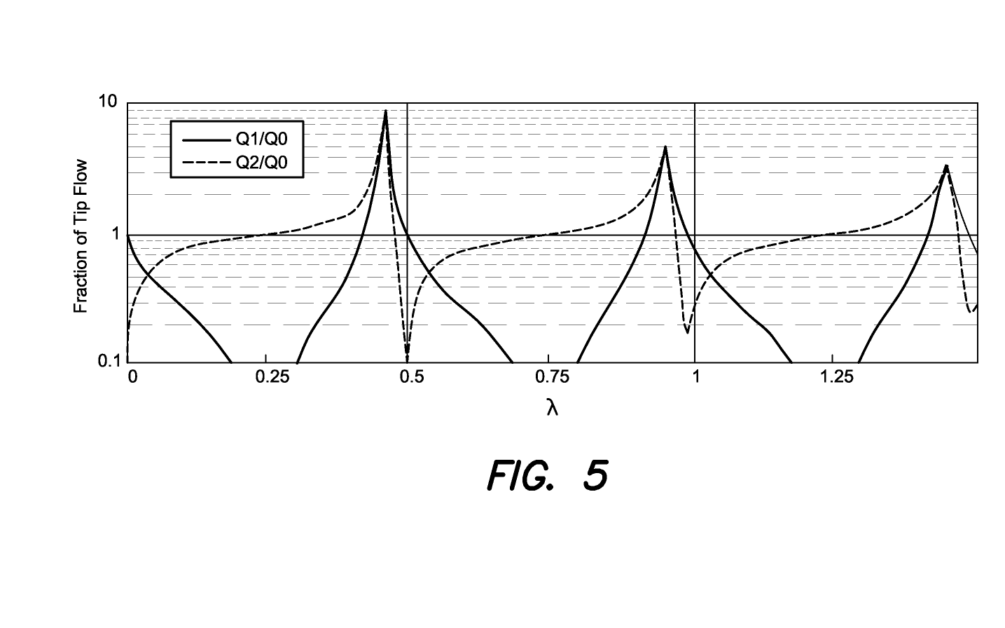

A graph of a water flow simulation for an ultrasonically vibrated needle is shown below, in FIG. 5. FIG. 5 clearly illustrates the levels of bi-directional flow through a needle port at various wavelengths λ. Again it is noted that the inventors believe it is the creation of bi-directional flow through the port, without external cavitation that allows for effective vitreous dissection without clogging the needle and without damaging delicate retinal tissue.

For simplicity, the simulations presumed that the back opening of the needle or cannula lumen transitioned into infinite space. In practice, the needle/cannula lumen will transition into a significantly larger lumen of the aspiration path, which will present its own acoustical load. However, because the cross-sectional area of the larger lumen is significantly larger than the cross-sectional area of the needle lumen, the residual acoustic effects are minimized and can be ignored.

FIG. 5 is a graph of a mathematical simulation showing the ratio of the displacement fluid flow (without any aspiration from a vacuum source) created by a vibrating needle in water from an inner surface at the distal tip 18 (Q0) passing out of the needle through the port 16 (Q2, solid line) or flow that moves down the cannula lumen, away from the port 16 (Q1, dotted line). That is to say Q0 is volumetric flow at the inner surface at the distal tip 18, Q2 is flow through the port 16, and Q1 is flow within the cannula lumen 14 away from the port 16. For a ratio of flow values of 1, the volume flow in a particular direction is equal to the volume flow from the inner surface at the distal tip 18. Where the ratio of flow is greater than 1, flow out of the port can exceed the tip flow in theory because of resonant effects. However, at ratio of flows greater than 1 where the amplitude of Q0 is high, cavitation bubbles will form at the nodes in the middle of the lumen, preventing the flow out of the port from exceeding the tip flow. Therefore, in practice, the Q1/Q0 and Q2/Q0 curves above 1 in FIG. 5 would degrade to values around 1. It can be seen that at the half, full, and wave and a half lengths (0.5, 1, and 1.5) Q2/Q0 is low. At the odd quarter of the drive frequency wavelengths (0.25, 0.75, 1.25), flow out the port 16 (Q2/Q0) is equal to the displacement flow, and flow down the shaft (Q1/Q0) is minimized. The odd quarter wavelengths are the optimal points, but it can also be seen that within about an eighth of the odd quarter drive frequency wavelength (about +/−0.125 wavelengths) the port flow affect is minimal and the device performance is expected to be stable.

For any of the example embodiments of FIGS. 2, 3, and 4 the cannula 12 may have an outer diameter of one of 23, 25, and 27 gauge so that the cannula may be inserted through an entry site alignment device (not shown) as is known. The water column length which, depending on the handpiece and needle constructing is typically one of the cannula lumen length 28 or the needle lumen length 44 may be between approximately 33 and 47 mm when the drive frequency is approximately 28 kHz.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A needle for use with an ultrasonic handpiece for dissecting and aspirating vitreous tissue, the needle comprising:
a cannula having a lumen extending from a port adjacent a cannula distal tip to a cannula proximal end;
wherein the cannula port has a cross-sectional area less than a cross-sectional area of the cannula lumen;
a hub attached to the cannula proximal end, the hub including structure for attachment to the ultrasonic handpiece;
wherein the hub includes a lumen extending through the hub, the hub lumen being coaxial and in communication with the cannula lumen, such that a needle lumen is formed from the cannula port to a proximal end of the hub; and
wherein a length of the needle lumen is approximately an odd quarter of a drive frequency wavelength of the ultrasonic handpiece and a cannula length measured from the cannula distal tip to a distal end of the hub is long enough to extend from an incision site across a posterior segment of an eye.

2. The needle of claim 1, wherein the cannula has an outer diameter of less than one of 23, 25, and 27 gauge.

3. The needle of claim 1, wherein the hub attachment structure are threads.

4. The needle of claim 1, wherein the needle lumen length is within about an eighth of the odd quarter drive frequency wavelength.

5. The needle of claim 4, wherein the needle lumen length is between approximately 33 and 47 millimeters when the drive frequency is approximately 28 kilohertz.

6. The needle of claim 1, wherein the needle lumen length is approximately 38 mm and the drive frequency is one of approximately 10 kilohertz and 30 kilohertz.

7. The needle of claim 1, wherein the drive frequency is determined by the equation f=(c/l)*{(2n−1)/4}, where c is approximately a phase speed of a wave in water, l is the needle lumen length, f is the drive frequency, and n is a positive integer.

8. The needle of claim 1, wherein the needle lumen length is determined by the equation l=(c/f)*{(2n−1)/4}, where c is approximately a phase speed of a wave in water, l is the needle lumen length, f is the drive frequency, and n is a positive integer.

9. A needle for use with an ultrasonic handpiece for dissecting and aspirating vitreous tissue, the needle comprising:
a cannula having a lumen extending from a port adjacent a cannula distal tip to a cannula proximal end;
wherein the cannula port has a cross-sectional area less than a cross-sectional area of the cannula lumen;
a hub attached to the cannula proximal end, the hub including structure for attachment to the ultrasonic handpiece;
wherein the hub includes a lumen extending through the hub, the hub lumen being coaxial and in communication with the cannula lumen; and
wherein a length of the cannula lumen is approximately an odd quarter of a drive frequency wavelength of the ultrasonic handpiece and a cannula length measured from the cannula distal tip to a distal end of the hub is long enough to extend from an incision site across a posterior segment of an eye.

10. The needle of claim 9, wherein the cannula has an outer diameter of less than one of 23, 25, and 27 gauge.

11. The needle of claim 9, wherein the hub attachment structure are threads.

12. The needle of claim 9, wherein the cannula lumen length is within about an eighth of the odd quarter drive frequency wavelength.

13. The needle of claim 12, wherein the cannula lumen length is between approximately 33 and 47 millimeters when the drive frequency is approximately 28 kilohertz.

14. The needle of claim 9, wherein the cannula lumen length is approximately 38 mm and the drive frequency is one of approximately 10 kilohertz and 30 kilohertz.

15. The needle of claim 9, wherein the drive frequency is determined by the equation f=(c/l)*{(2n−1)/4}, where c is approximately a phase speed of a wave in water, l is the cannula lumen length, f is the drive frequency, and n is a positive integer.

16. The needle of claim 9, wherein the needle lumen length is determined by the equation 1=(c/f)*{(2n−1)/4}, where c is approximately a phase speed of a wave in water, l is the cannula lumen length, f is the drive frequency, and n is a positive integer.

17. A needle for use with an ultrasonic handpiece for dissecting and aspirating vitreous tissue, the needle comprising:
a cannula having a lumen extending from a port adjacent a cannula distal tip to a cannula proximal end;
wherein the cannula port has a cross-sectional area less than a cross-sectional area of the cannula lumen;
the cannula for attachment to a distal end of the ultrasonic handpiece, such that the cannula lumen is in communication with an aspiration path formed in the ultrasonic handpiece, wherein the aspiration path has a cross-sectional area significantly larger than the cannula lumen cross-sectional area; and
wherein a length of the cannula lumen is approximately an odd quarter of a drive frequency wavelength of the ultrasonic handpiece and a cannula length measured from the cannula distal tip to the distal end of the ultrasonic handpiece is long enough to extend from an incision site across a posterior segment of an eye.

18. The needle of claim 17, wherein the cannula has an outer diameter of less than one of 23, 25, and 27 gauge.

19. The needle of claim 17, wherein the hub attachment structure are threads.

20. The needle of claim 17, wherein the cannula lumen length is within about an eighth of the odd quarter drive frequency wavelength.

21. The needle of claim 20, wherein the cannula lumen length is between approximately 33 and 47 millimeters when the drive frequency is approximately 28 kilohertz.

22. The needle of claim 17, wherein the cannula lumen length is approximately 38 mm and the drive frequency is one of approximately 10 kilohertz and 30 kilohertz.

23. The needle of claim 17, wherein the drive frequency is determined by the equation f=(c/l)*{(2n−1)/4}, where c is approximately a phase speed of a wave in water, l is the cannula lumen length, f is the drive frequency, and n is a positive integer.

24. The needle of claim 17, wherein the cannula lumen length is determined by the equation 1=(c/f)*{(2n−1)/4}, where c is approximately a phase speed of a wave in water, l is the cannula lumen length, f is the drive frequency, and n is a positive integer.
